(19) United States
(12) Patent Application Publication
(10) Pub. No.: US 2007/0087029 A1
(43) Pub. Date: Apr. 19, 2007

(54) Localized delivery to the lymphatic system

(76) Inventor: Syamasundar V. Pakala, Blaine, MN (US)

Correspondence Address: PILLSBURY WINTHROP SHAW PITTMAN, LLP, P.O. BOX 10500, MCLEAN, VA, 22102, US

(21) Appl. No.: 11/580,534

(22) Filed: Oct. 13, 2006

Related U.S. Application Data
(60) Provisional application No. 60/727,090, filed on Oct. 14, 2005.

(51) Int. Cl. A61K 38/22; A61K 38/18; A61F 2/02

(52) U.S. Cl. 424/423; 514/12

(57) ABSTRACT

The present invention provides methods for the localized delivery of an agent to the lymphatic system, including localized delivery to a lymphatic capillary bed.

### CROSS REFERENCE TO RELATED APPLICATIONS

This application claims benefit of priority to U.S. Provisional Patent Application Ser. No. 60/727,090 filed Oct. 14, 2005, hereby incorporated by reference in its entirety.

### FIELD OF THE INVENTION

The present disclosure relates generally to localized delivery of an agent to tissues. In particular, localized delivery of an agent to the lymphatic system, including localized delivery to a lymphatic capillary bed.

### BACKGROUND OF THE INVENTION

The transplantation of tissue between individuals of the same species or between individuals from different species results in the activation of the host immune system and the rejection of the transplanted tissue. Successful transplantation thus relies on suppressing the transplant recipient's immune system to prevent the rejection of the transplanted tissue. Currently, such suppression is achieved with the systemic administration of one or more immunosuppressive agents. However, the systemic administration of such immunosuppressive agents has the harmful side effects of disabling the recipient's ability to respond to normal pathogenic exposures and severely limiting the recipient's ability to combat cancers. Further, many immunosuppressive agents are toxic to the liver and the kidneys and cannot be administered systemically for extended periods of time without significant damage to these organs. Thus, there is a need for improved methods for the localized delivery of agents such as immunosuppressive agents.

### SUMMARY OF THE INVENTION

The present invention provides a method for the localized delivery of an agent to the lymphatic system of a subject, the method including providing the subject with a delivery device and delivering the agent via the delivery device, wherein the agent is delivered into a lymphatic capillary bed.

In another aspect, the present invention provides a method for the localized delivery of an agent to the lymphatic system of a subject, the method including providing a delivery device to the subject and delivering the agent via the delivery device, wherein the delivery of the agent by the delivery device is active, variable, implantable, or a combination thereof, and wherein the agent is delivered into a lymphatic capillary bed.

In another aspect, the present invention provides a method for limiting the distribution of an agent in a subject's body, the method including providing the subject with a delivery device and delivering the agent via the delivery device, wherein the agent is delivered into a lymphatic capillary bed.

In some embodiments of the present invention, the delivery device has a variable delivery rate. In some embodiments of the present invention, delivery of the agent by the device is active. In some embodiments of the present invention, the delivery device is implanted in the subject. In some embodiments of the present invention, the agent is delivered to one or more lymph nodes of the lymphatic capillary bed. In some embodiments of the present invention, the delivery device is located subcutaneously. In some embodiments of the present invention, the delivery device is located intraperitoneally. In some embodiments of the present invention, the agent is delivered to the surface of a solid organ. In some embodiments of the present invention, the agent is delivered to the peritoneal membrane.

In some embodiments of the present invention, the agent is an immunomodulator. In some embodiments of the present invention, the agent is an immunosuppressant. In some embodiments of the present invention, the agent is a hormone or growth factor. In some embodiments of the present invention, the agent is an angiogenic factor. In some embodiments of the present invention, delivery is to a site from which a tumor has been surgically excised.

In some embodiments of the present invention, the subject has been the recipient of a cell, tissue, or organ transplant. The cell, tissue, or organ transplant may be located within the lymphatic capillary bed. The cell, tissue, or organ transplant may be, for example, pancreatic islet cells, cardiac cells, muscle cells, a heart, a heart valve, liver, lung, kidney, or pancreas. The cell, tissue, or organ transplant may be genetically modified to express a therapeutic agent.

In some embodiments of the present invention, the delivery device delivers a cell, tissue or organ transplant. The cell, tissue, or organ transplant may be located within the lymphatic capillary bed. The cell, tissue, or organ transplant may be, for example, pancreatic islet cells, cardiac cells, muscle cells, a heart, a heart valve, liver, lung, kidney, or pancreas. The cell, tissue, or organ transplant may be genetically modified to express a therapeutic agent.

Unless otherwise specified, “a,” “an,” “the,” and “at least one” are used interchangeably and mean one or more than one.

### BRIEF DESCRIPTION OF THE FIGURES

FIGS. 1A-1C. Acute, subcutaneous injection of isosulfan blue. FIG. 1A shows the location of two different injection sites. FIG. 1B shows that injection of dye one centimeter above the hip joint covers the inguinal bed. FIG. 1C shows that injection of dye one centimeter above the hip joint covers the popliteal bed.

FIGS. 2A-2C. Acute injection into the peritoneal wall. FIG. 2A shows the location of the injection site into the peritoneal wall, one centimeter above the hip joint. FIG. 2B shows that injection of dye into the peritoneal wall one centimeter above the hip joint covers the inguinal bed. FIG. 2C shows that injection of dye into the peritoneal wall one centimeter above the hip joint covers the popliteal bed.

FIGS. 3A-3B. Sites for implantation of Alzet® pump for chronic/continuous delivery of a dye. FIG. 3A shows the location for subcutaneous (SQ) implantation. FIG. 3B shows the site for implantation into the peritoneal wall (IP).

FIG. 4. Peroxidase activity in lymph nodes after administration subcutaneously (“SC”) and above the peritoneal membrane (“IP”). The popliteal (“PoLN”) and the para-aortic lymph nodes (labeled ParaLN) show higher peroxidase activity as compared to control lymph node (unlabelled, extreme right) with some activity in the mesenteric (labeled MLN) and the inguinal lymph nodes labeled (ILN). “N-2” represents animal N-2. “N-11” represents animal N-11.

FIGS. 5A-5B. Continuous delivery of India ink. FIG. 5A shows the catheter with a spacer capable of retarding the occlusion of the catheter. FIG. 5B shows site of catheter implantation. FIG. 5C identifies the primary draining site as the ipsilateral para-aortic lymph node (ParaLN); no staining was observed in the contralateral para-aortic lymph node (C-Para-LN).

FIG. 6. Histology of lymph nodes with local, chronic administration of india ink to the intraperitoneal (IP) wall. The nondraining popliteal lymph node (LN) shows no carbon deposition while the draining para-aortic lymph node (LN) shows discrete patches of carbon black.

FIG. 7. Placement of catheters for localized delivery.

FIGS. 8A and 8B. Antibody has minimal effect on peripheral blood, thus demonstrating no systemic effect of continuous delivery of local immunosuppression. As shown for both week one (FIG. 8a) and week four (FIG. 8B), there was no difference between the untreated (groups 9 and 10) and treated (groups 1-8).

FIG. 9. Local continuous delivery of immunosuppressive compounds does not affect relative ratios of lymphocytes in circulating blood. The data shown represent percentage of CD3 T lymphocytes in peripheral blood. Each data set is the average of three animals.

FIGS. 10A-10E. Effect of local delivery of rat anti-lymphocyte serum (RALS) on cell number. Cell Counts of lymphocytes extracted from different lymphoid organs. FIG. 10A represents cell counts from untreated control animals. FIG. 10B represents cells counts from subcutaneous high dose RALS treated animals. FIG. 10C represents cell counts from subcutaneous low dose RALS treated animals. FIG. 10D represents cell counts from IP-wall high dose RALS treated animals. FIG. 10E represents cell counts from IP-wall low dose RALS treated animals. FIG. 10A presents data from two animals per group. FIG. 10B-10E present data from three animals per group.

FIG. 11A-11E. Effect of local delivery of rat anti-lymphocyte serum (RALS) on cell proliferation. Proliferative response of lymphocytes stimulated with 1000 micrograms (μg) of ovalbumin for 72 hours. Lymphocytes were extracted from different lymphoid organs as indicated and plated concentration of 3×10⁶ cells per well. Proliferative responses were determined by thymidine ³[H] incorporation. FIG. 11A represents untreated control animals. FIG. 11B represents subcutaneous high dose RALS treated animals. FIG. 11C represents subcutaneous low dose RALS treated animals. FIG. 11D represents IP-wall high dose RALS treated animals. FIG. 11E represents IP-wall low dose RALS treated animals. FIG. 11A presents data from two animals per group. FIG. 11B-11E present data from three animals per group.

FIG. 12A-12E. Effector cytokine responses. The cytokine secretion patterns of lymphocytes stimulated with pg of ovalbumin for 36 hours are shown. Lymphocytes were extracted from the different lymphoid organs indicated and plated at a concentration of 3×10⁶ cells per well. FIG. 12A represents untreated control animals. FIG. 12B represents subcutaneous high dose RALS treated animals. FIG. 12C represents subcutaneous low dose RALS treated animals. FIG. 12D represents IP-wall high dose RALS treated animals. FIG. 12E represents IP-wall low dose RALS treated animals. FIG. 12A presents data from two animals per group. FIG. 12B-12E present data from three animals per group.

FIG. 13. Local immunosuppression preferentially affects immune responses in the targeted lymph nodes. The data points are readings from individual animals for control targeted (∘), treatment targeted (●), control non-targeted (□), and treatment non-targeted (▪). Bars represent the mean.

### DETAILED DESCRIPTION OF ILLUSTRATIVE EMBODIMENTS OF THE PRESENT INVENTION

The present invention provides methods for the localized delivery of an agent to the lymphatic system, including localized delivery of an agent to a lymphatic capillary bed. Localized delivery (also referred to herein as local delivery) targets the delivery of an agent to a desired location. Localized delivery limits the distribution of an agent in a subject's body, for example, limiting distribution to one or more lymphatic capillary beds, one or more lymph nodes, including primary lymph nodes and/or secondary lymph nodes, and/or other lymphatic tissues and organs. Localized delivery can focus or limit the delivery of an agent to a target location within the body.

Such localized delivery provides many advantages over the systemic administration of an agent. The localized delivery of an agent lowers the overall systemic dose exposure to the agent and minimizes the side effects of the broad, systemic administration of an agent. Localized delivery can provide for the continuous delivery of an agent to a site within the body. Localized delivery allows for the administration of a high dose of an agent without the associated toxicities noted with systemic administration. Such toxicities can include, for example, nephrotoxicity and/or hepatotoxicity.

The Lymphatic System

With the present invention, the placement of a delivery device in a particular location within the lymphatic system results in the localized delivery of an agent to a lymphatic capillary bed, also referred to herein as a lymphatic drainage bed, draining this location within the lymphatic system. The localized delivery of an agent to a lymphatic capillary bed may result in the delivery of the agent to the first lymph nodes draining the lymphatic capillary bed, also referred to as “primary” lymph nodes. In some cases, the localized delivery of an agent may also result in the delivery of the agent to additional lymph nodes downstream of the primary lymph nodes, also referred to as “secondary” lymph nodes. In some aspects of the invention the agent may eventually enter the blood stream and be delivered systemically, but this delivery is at a significantly reduced concentration, and after localized delivery of the agent.

The lymphatic system is a part of the immune system, protecting the body against infection and invasion by foreign organisms. Lymphocytes and macrophages patrol most of the body's tissues for invading viruses, bacteria, tumor cells, foreign proteins, toxins, damaged and dying cells, and foreign cells, including, foreign tissue grafts. Lymph vessels communicate with most tissues, transporting the lymph fluid that carries the immune cells to the lymph nodes and lymphatic organs, such as the spleen and thymus. The lymphatic vessels, also referred to as lymphatics, are a network of thin tubes that branch, like blood vessels, into tissues throughout the body. In mammals, including humans, most tissues and organs are drained by the lymphatic system.

Unlike the circulatory system, the lymphatic system is not closed and has no central pump. The lymphatic system forms a one-way flow system towards the heart. Through this system flows lymph. Flow begins in narrow lymphatic capillaries. An elaborate network of lymph capillaries drains interstitial fluid from the tissues. Lymphatics enter all tissues except epithelia, brain, spinal cord, and bone marrow. A few connective tissues, such as cartilage and the cornea, have no blood vessels and also lack lymphatics. Lymph moves slowly and under low pressure. The capillaries have one-way valves, which ensure flow is only in one direction. Lymph is further moved through the system by means of compression caused by general skeletal muscle movements, including pulmonary inspiration. Lymphatic capillaries are ten to fifty micrometers in diameter, and are referred to as the initial (or terminal) lymphatics. They start from a blind sac, or from anastomising vessels. The endothelium is a single layer, with an incomplete basement membrane. They possess gap junctions that are highly permeable to plasma proteins and large particles, including, for example, carbon particles, pathogens, such as viruses, bacterial cells, and parasites, cells, including, for example, immune cells and tumor cells, and cellular debris. The methods of the present invention may be used for the localized delivery of an agent to one or more lymphatic capillary beds.

Lymph flows from capillaries into collecting lymphatics where it encounters the first of many lymph nodes. Lymph nodes filter lymph, with an internal honeycomb of connective tissue filled with lymphocytes that collect and destroy bacteria and viruses. Lymph nodes also produce lymphocytes and antibodies. When the body is fighting an infection, these lymphocytes multiply rapidly and produce a characteristic swelling of the lymph nodes. Approximately twenty-five billion different lymphocytes migrate through each lymph node every day. Lymph is transported to progressively larger lymphatic vessels culminating in the right lymphatic duct (for lymph from the right upper body) and the thoracic duct (for the rest of the body). These ducts drain into the circulatory system at the right and left subclavian veins, near the shoulders.

Along the network of lymphatic vessels are a series of various lymphatic tissues and organs, including lymphatic nodules, Peyer's patches, tonsils, lymph nodes, the thymus, and the spleen. Lymphatic nodules are transient clusters of lymphocytes that form at sites of infection and then disappear. No capsule or external covering separates nodules from the surrounding cells and fluids, which percolates directly into the nodules. Peyer's patches are larger nodular clusters of lymphocytes located in the walls of the intestines and the tonsils are pockets of nodular tissue enfolded into the mucosa of the pharynx. Peyer's patches and the tonsils are situated to intercept antigens from the digestive and respiratory tracts, respectively. The methods of the present invention may be used for the localized delivery of an agent to lymphatic nodules, Peyer's patches, and/or the tonsils.

Lymph nodes encapsulate many lymphatic nodules within a tough capsule and are supplied with blood vessels and lymphatics. Lymph nodes filter the lymph delivered to them by lymphatic vessels. Thus, lymph nodes filter the lymph draining from the lymphatic capillary bed in which the lymph node is situated. The methods of the present invention may be used for the localized delivery of an agent to one or more draining lymph nodes.

Clusters of lymph nodes are found in various anatomical regions and the methods of the present invention may be used for the localized delivery of an agent to any one or more of these lymphatic regions. For example, clusters of lymph nodes are found in the underarm (the axillary lymph nodes), the groin (the inguinal lymph nodes), the neck (the cervical lymph nodes), the chest (pectoral lymph nodes), and the abdomen (the iliac lymph nodes). Other lymphatic clusters include, but are not limited to, the popliteal lymph nodes, parasternal lymph nodes, lateral aortic lymph nodes, paraaortic lymph nodes, submental lymph nodes, parotid lymph nodes, submandibular lymph nodes, intercostal lymph nodes, diaphragmatic lymph nodes, pancreatic lymph nodes, citerna chili, lumbar lymph nodes, sacral lymph nodes, obturator lymph nodes, mesenteric lymph nodes, mesocolic lymph nodes, gastric lymph nodes, hepatic lymph nodes, and splenic lymph nodes. The methods of the present invention may be used for the localized delivery an agent to any one or more of these lymphatic regions.

Lymph is a colorless, watery fluid originating from interstitial fluid. Lymph originates as blood plasma lost from the capillary beds of the circulatory system, which leaks out into the surrounding tissues. Although the capillaries of the circulatory system lose only about 1% of the volume of the fluid that passes through them, so much blood circulates that the cumulative fluid loss in the average human body is about three liters per day. The lymphatic system collects this fluid by diffusion into lymph capillaries, and returns it to the circulatory system. Once within the lymphatic system the fluid is called lymph, and has almost the same composition as the original interstitial fluid.

The spleen, lymph nodes, and accessory lymphoid tissue (including the tonsils and appendix) are the secondary lymphoid organs. These organs are made up of a scaffolding of connective tissue that supports circulating B- and T-lymphocytes and other immune cells, including, for example, macrophages, dendritic cells, and eosinopils. When microorganisms invade the body or the body encounters other antigens, the antigens are typically transported from the tissue to the lymph. The lymph is carried in the lymph vessels to regional lymph nodes. In the lymph nodes, the macrophages and dendritic cells phagocytose antigens, process antigens, and present antigens to lymphocytes, which can then start producing antibodies or serve as memory cells to recognize the antigens again in the future. Lymph and lymphoid tissue thus contain antibodies and immune cells.

Lymphocytes are produced by stem cells in the bone marrow and then migrate to either the thymus or bone marrow where they mature. T-lymphocytes undergo maturation in the thymus (hence their name), and B-lymphocytes undergo maturation in the bone marrow. After maturation, both B- and T-lymphocytes circulate in the lymph and accumulate in secondary lymphoid organs, where they await recognition of antigens.

The methods of the present invention may be used for the delivery of an agent to afferent and/or efferent lymphatic vessels. As used herein, “afferent” lymphatic vessels bring lymph to a lymph node and “efferent” lymphatic vessels conduct lymph away from a lymph node.

As used herein, “ipsilateral” means situated on, pertaining to, or affecting the same side. As used herein, “contralateral” means situated on, pertaining to, or affecting the opposite side.

Delivery Devices

Any of a wide variety of means may be used for the localized delivery of an agent to a lymphatic capillary bed. An agent may be delivered by any known or future developed mechanism. Localized delivery of an agent to a lymphatic capillary bed may be by active or passive delivery.

As used herein, the passive delivery of the agent is delivery accomplished without active intervention. Examples of passive delivery include, but are not limited to, administration in an implanted capsule that permits the agent within the capsule to transfer outside of the capsule wall by diffusion and/or with the dissolving of the capsule wall (see, for example, U.S. Pat. Nos. 5,106,627 and 5,639,275), administration as a time-released formulation, administration as a coating on a delivery device, or administration by the deposition of the agent in a depot formulation.

A potential drawback with such passive approaches is that drug delivery cannot be controlled after implantation of the formulation. Thus, in some aspects of the present invention, local delivery of an agent to a lymphatic capillary bed is not by passive delivery. In some aspects of the present invention, local delivery of an agent to a lymphatic capillary bed is not by administration as an implanted capsule. In some aspects of the present invention, local delivery of an agent to a lymphatic capillary bed is not by administration as a time released formulation. In some aspects of the present invention, local delivery of an agent to a lymphatic capillary bed is not by administration as a coating on a delivery device. In some aspects of the present invention, local delivery of an agent to a lymphatic capillary bed is not by administration by the deposition of the agent in a depot formulation.

As used herein, active delivery of an agent is accomplished by active efforts or intervention, including, for example, by an infusion pump. The medical device industry produces a wide variety of devices that may be utilized for the active delivery of an agent. For example, any of a variety of electrical, mechanical, chemical, electrochemical, osmotic, or electro-osmotic devices may be utilized. A delivery device may be implanted or may be placed external to the subject.

A delivery device may be a pump device. Non-limiting examples of pump devices include fixed-rate pumps, selectable rate pumps, variable rate pumps, and the like. Any pumping mechanism may be used. For example, the pump may be any of an osmotic pump, a piston pump, a peristaltic pump, and the like. The pump device may be programmable. Each of the aforementioned pump systems may comprise a reservoir for housing a fluid composition comprising an agent.

In some aspects, the local delivery of an agent to a lymphatic capillary bed may be accomplished with the use of an implantable device. For example, an implantable drug infusion device may be used. An implantable infusion device is designed to be implanted into a patient's body, to administer an infusion media to the patient. A variety of such implantable drug infusion devices are well known in the art. These devices may include a medication reservoir within a housing. Some form of fluid flow control is also provided to control or regulate the flow of fluid medication from the reservoir to the outlet of the device for delivery of the medication to the desired location in a body, usually through a catheter. An implantable drug infusion device may provide for the delivery of an agent at a regulated dosage. An implantable drug infusion device may deliver an agent at a constant rate or a variable rate. The rate of delivery may be the same as the rate of local lymphatic drainage, or may be a rate that is greater than or less than the rate of local lymphatic drainage. An implantable drug infusion device may provide for the delivery of an agent at a programmable dosage, including delivery at a programmable long-term dosage.

In some aspects, steps may be taken to minimize and/or prevent the occlusion of the outlet of the delivery device. Such occlusion may be the result of, for example, tissue ingrowth, clotting, fibrosis, and/or scarring. Steps taken may include, but are not limited to, adding a spacer or stent to the outlet of the delivery device and/or coating various surfaces of the device. Surfaces of the delivery device may be coated, for example, with one or more anticoagulants, one or more immune modulators, one or more anti-inflammatory agents, and various combinations thereof.

The infusion of the agent delivered by an implantable drug infusion device may be categorized as either active infusion or passive infusion (see, for example, U.S. Pat. No. 6,878,135). Active drug infusion devices feature a pump or a metering system to deliver the drug. An active drug infusion device may be programmable. An example of such an active drug infusion device currently available is the Medtronic SynchroMed™ programmable pump. Such pumps typically include a drug reservoir, a peristaltic pump that pumps the drug out from the reservoir, and a catheter port to transport the pumped out drug from the reservoir via the pump to a patient's anatomy. Such devices also typically include a battery to power the pump as well as an electronic module to control the flow rate of the pump. The Medtronic SynchroMed™ pump further includes an antenna to permit the remote programming of the pump.

Passive drug infusion devices, in contrast, do not feature a pump, but rather rely upon a pressurized drug reservoir and some form of fluid flow control to deliver the drug. An example of such a device includes the Medtronic IsoMed™ device. This device delivers the drug into the patient through the force provided by a pressurized reservoir.

The infusion rate of the drug delivery device may be programmed to be variable over time. The rate is usually controlled by certain components in the pump. The controlled infusion rate is often further set by using an external device or programmer to transmit instructions for the controlled infusion into the pump. The controlled infusion may be variable as time passes according to the needs of the patient. The instructions provided to the pump to control the infusion rate of the drug pump are typically determined by a medical person. In some cases the patient is able to provide the instructions to the pump via an external patient-programming device. In contrast, fixed rate pumps usually cannot be programmed and are only capable of constant infusion rate.

The methods of the present invention may be applicable for any suitable subject. Suitable subjects include, but are not limited to, animals such as, but not limited to, humans, non-human primates, rodents, dogs, cats, horses, pigs, sheep, goats, cows, or birds. A subject may be a patient undergoing medical treatment.

The methods of the present invention are suitable for a variety of medical objectives, including therapeutic, prophylactic, or diagnostic. As used herein, “treating” a condition or a subject includes therapeutic, prophylactic, diagnostic treatments, or a combination thereof.

Agents

Any of a wide variety of agents may be delivered by the methods of the present invention. For example, an agent delivered by the methods of the present invention may be an immunosuppressive agent. Examples of immunosuppressive agents to be delivered by the methods of the present invention include, for example, mycophenolate moefetil (also known as CellCept®), cyclosporine (also known as Sandimmune® and NEoral®), corticosteroids, azathiaprine (also known as Imuran®), chlorambucil (Leukeran®), cyclophosphamide (Cytoxan® and Fludara®), FK506, rapamycin, tacrolimus, cytarabine, mercaptopurine, inactivating ligands, such as, for example, CTLA4Ig, and immunosuppressive antibodies. Immunosuppressive antibodies include, but are not limited to, anti-lymphocyte serum (such as, for example, antilymphocyte immunoglobulin (ALG), antithymocyte immunoglobulin (ATG), antilymphocyte serum, antithymocyte serum, lymphocytic antiserum, thymitic antiserum, and ATGAM), anti-CD3 antibodies (such as Muromonab-CD3), anti-CD25 antibodies, anti-CD40 ligand antibody, anti-CD40 antibody, anti-CD30 antibody, anti-OX40 antibody, and anti-CD28 antibody (see, for example, U.S. Patent Application No. 2002/0006403 A1), anti-cytokine antibodies, such as, for example, anti-TNF antibodies and anti-IFN antibodies. ATGAM is a lymphocyte-selective immunosuppressant as is demonstrated by its ability to reduce the number of circulating, thymus-dependent lymphocytes that form rosettes with sheep erythrocytes. This anti-lymphocytic effect is believed to reflect an alteration of the function of the T-lymphocytes, which are responsible in part for cell-mediated immunity and are involved in humoral immunity.

Other immunosuppressive agents include, but are not limited to, azathioprine or azathioprine sodium, basiliximab, cyclosporin or cyclosporine (cyclosporin A), daclizumab (dacliximab), glatiramer or glatiramer acetate, mycophenolate, mycophenolate mofetil (MMF), mycophenolate morpholinoethyl, mycophenolic acid, tacrolimus (FK506), anhydrous tacrolimus, tacrolimus monohydrate, sirolimus, interferon α-2a, recombinant interferon α (rIFN-A or IFLrA), brequinar, brequinar sodium, cyclophosphamide, cyclophosphamide monohydrate, anhydrous cyclophosphamide, dactinomycin, actinomycin C, actinomycin D, meractinomycin, daunorubicin, daunorubicin hydrochloride, daunomycin hydrochloride, rubidomycin hydrochloride, doxorubicin, doxorubicin hydrochloride, adriamycin, adriamycin hydrochloride, fluorouracil, gusperimus, gusperimus hydrochloride, inolimomab, leflunomide, mercaptopurine, mercaptopurine monohydrate, purinethiol, anhydrous mercaptopurine, methotrexate, methotrexate sodium, methotrexate disodium, alpha-methopterin, amethopterin; mustine, mustine hydrochloride, chlormethine hydrochloride, chlorethazine hydrochloride, mechlorethamine hydrochloride, nitrogen mustard (mustine), mizoribine, vinblastine, vinblastine sulfate, vincaleukoblastine sulphate, capecitabine, carmofur, epirubicin, epirubicin hydrochloride, idarubicin, idarubicin hydrochloride, mafosfamide, menogaril, mitozantrone, mitozantrone hydrochloride, mitoxantrone hydrochloride, pirarubicin, tepirubicin, piroxantrone, piroxantrone hydrochloride, anthrapyrazole hydrochloride, oxantrazole hydrochloride, teloxantrone, teloxantrone hydrochloride, thioguanine, anhydrous thioguanine, thiguanine hemihydrate, tioguanine, trofosfamide, trilophosphamide, trophosphamide, uramustine, uracil mustard, vincristine, vincristine sulphate, leurocristine sulphate, vindesine, vindesine sulfate, desacetyl vinblastine amide sulfate, vinorelbine, vinorelbine tartrate, vinorelbine ditartrate, zorubicin, zorubicin hydrochloride, rapamycin, thalidomide, clofazimine, fludarabine, guanosine arabinoseide, cytosine arabinoseide, prednisone, glucocorticoids, and a pharmacologically or physiologically acceptable salt of any of the foregoing.

An agent to be delivered by the methods of the present invention may be an immunostimulatory agent. Examples of immunostimulatory agents to be delivered by the methods of the present invention include, but are not limited to, immunostimulatory antibodies such as anti-CD40 antibodies, anti-CD137 antibodies, and anti-CD134 antibodies, and cytokines, including, for example, IL-2, IL-12, and IL-18.

An agent delivered by the methods of the present invention may be an antineoplastic agent. Examples of antineoplastic agents to be delivered by the methods of the present invention include, but are not limited to, nitrogen mustard (Mustargen), doxorubicin hydrochloride (Adraimycin®), methotrexate, cis-platin, carboplatin, taxols, anti-tumor antibodies conjugated to toxins such as ricin, compounds and antibodies that prevent vascularization, such as anti-VEGF antibodies, and siRNA to disrupt cancer proliferation.

Agents to be delivered by the methods of the present invention include antigens and vaccines. For example, a tumor vaccine may be delivered by the methods of the present invention. Tumor vaccines containing a specific protein of the tumor cell can be used to stimulate an immune response. Different types of vaccines are used to treat different types of cancer, including, for example, melanoma, myeloma, pancreatic cancer, prostate cancer, breast cancer, colon cancer, kidney cancer, lung cancer, or lymphoma. With the methods of the present invention, a tumor vaccine may be administered to the lymphatic capillary bed in which the tumor is located and/or the lymphatic capillary bed that drains the tumor.

An agent delivered by the method of the present invention may be a biologic. As used herein, biologics are substances that are living matter or derived from living matter intended to have a therapeutic effect, including, for example, lymphocytes, genetically modified cells, stem cells, platelets, hormones, antibodies, proteins, biologically produced chemicals, and the like. Examples of biologics to be delivered by the methods of the present invention include, but are not limited to, Herceptin® (Trastizumab, an anti-HER2 monoclonal antibody), Avastin™ (bevacizumab, an anti-VEGF monoclonal antibody), Tituxan® (tituximab, an anti-CD20 monoclonal antibody) and Tarceva™ (erlotinib, a small molecule HER1/EGFR inhibitor).

Agents delivered by the methods of the present invention also include dyes, detectable markers, radiolabeled pharmaceuticals, such as, for example, technetium-99m sulfur colloid, hormones, adjuvants, pain medications, antibiotics, such as, for example, penicillin or tetracycline, corticosteroids, such as hydrocortisone or betamethasone, nonsteroidal antiinflammatories, such as fluriprofen, ibuprofen, or naproxen, antivirals, such as acyclovir or valcyclovir, chemotherapeutic agents, angiogenic or lymphangiogenic molecules, including, for example, angiopoietin, such as, for example, angiopoietin-1, angiopoietin-2, and angiopoietin-3, vascular endothelial growth factor (VEGF), such as, for example, VEGF-A, VEGF-C, and human recombinant VEGF-C (see Szuba et al., FASEB J. 2002 16(14):1985-7), and the like.

An agent to be delivered by the method of the present invention may be a genetic material. Genetic materials are substances intended to have a direct or indirect genetic therapeutic effect and include, for example, genetic vectors, genetic regulator elements, genetic structural elements, DNA, small interfering RNA (siRNA), and the like.

As used herein an agent includes a single agent or the combination of more than one agent. An agent may be formulated as a pharmaceutical composition configured to function in an implanted environment with characteristics such as stability at body temperature to retain therapeutic qualities, concentration to reduce the frequency of replenishment, and the like. When more than one agent is delivered, the agents may be delivered simultaneously and/or sequentially.

The precise amount of any agent delivered by the methods of the present invention will vary according to factors known in the art including but not limited to the physical and chemical nature of the agent, the intended dosing regimen, the state of the subject's immune system (for example, suppressed, compromised, or stimulated), and the species to which the formulation is being administered. Accordingly, it is not practical to set forth generally the amount that constitutes an amount of agent effective for all possible applications. Those of ordinary skill in the art, however, can readily determine the appropriate amount with due consideration of such factors.

The term “a therapeutically effective amount” means an amount of an agent sufficient to induce a therapeutic effect, such as, for example, suppression of an immune response, demonstration of antiviral or antitumor activity, or the reduction or elimination of neoplastic cells. The amount of an agent that will be therapeutically effective in a specific situation will depend on such things as the activity of the particular compound, the dosing regimen, the application site, the particular formulation and the condition being treated. As such, it is generally not practical to identify specific administration amounts herein; however, those skilled in the art will be able to determine appropriate therapeutically effective amounts based on the guidance provided herein and information available in the art pertaining to these agents.

Applications

The methods of the present invention may be used in a wide variety of applications. For example, the methods of the present invention may be used for the localized delivery of one or more of the various immunosuppressive agents administered to a transplant recipient to prevent the rejection of transplanted cells, tissues and/or organs. Localized delivery of an immunosuppressive agent may be used in to enhance the survival of transplanted cells, tissue, and/or organs. Localized delivery of an immunosuppressive agent may be used in the prevention and/or treatment of graft versus host (GVH) disease. Localized delivery may be to one or more regions of the lymphatic system draining the site(s) of the transplanted cells, tissue and/or organs. Localized delivery of an immunosuppressive agent may be used to minimize the side effects observed with the systemic administration of such immunosuppressive agents. The methods of the present invention may be used with autologous transplants, allogeneic transplants (also referred to as an allograft), or xenogeneic transplants (also referred to as a xenograft). A xenograft includes, for example, cells, tissues or organs of porcine origin. Transplanted organs include, for example, kidney, heart, liver, lung, small intestine, and pancreas. One or more intact organs may be transplanted. Transplanted tissues can include, for example limbs, and heart valves. Transplanted cells include, for example, myocardial cells, pancreatic islet cells, liver cells, spleen cells, bone marrow cells, and stem cells.

The methods of the present invention may be used in the treatment of diabetes and in diabetes cell therapy. Type I diabetes, also referred to as insulin dependant diabetes mellitus, is caused by the irreversible destruction of the insulin producing beta cells in the islets of Langerhans. While insulin therapy converts this life threatening disease into a chronic disease, long-term complications of the loss of beta cells, including the loss of glucose counter-regulation, have prompted the transplantation of cadaveric islets as a beta cell replacement therapy. Currently, the Edmonton protocol is commonly used, where the hepatic infusion of cadaveric islets, along with the systemically administration of immunosuppressive agents, leads to 80% of the patients being insulin free for one year. However, a longer-term analysis reveals that the five-year success rate drops to 25%. One potential reason why islet transplants fail after the first year is that the patients are “weaned off” immunosuppressive agents. This is primarily done to restore some immune functionality against normal pathogens and antigens and to combat the severe nephrotoxic and hepatotoxic nature of the immunosuppressive drugs.

The methods of the present invention, for the localized delivery of immunosuppressive agents to a lymphatic bed, may be used in conjunction with such methods for the treatment of diabetes. One or more immunosuppressive agents may be administered to the capillary beds draining the anatomical sites in which the islet cells have been transplanted or injected. Transplanted islet cells may be allogeneic or xenogeneic. Transplanted islet cells may be cadaveric.

The methods of the present invention, for the localized delivery of immunosuppressive agents to a lymphatic bed, may be used in conjunction with the delivery of syngeneic, allogeneic or xenogeneic myoblasts to the heart. Such myoblast cells may be genetically modified.

The methods of the present invention may be used for the localized delivery of an agent for the treatment of an autoimmune disease, including, for example, autoimmune chronic active hepatitis, Crohn's disease, systemic lupus erythematosus (SLE), including, for example, the renal effects of SLE, psoriasis, diabetes mellitus, multiple sclerosis, rheumatoid arthritis, systemic sclerosis, dermatomyositis, polymyositis, Sjogren's syndrome, polyarteritis nodosa, or vasculitis.

The methods of the present invention may be used for the localized delivery or installation of genetically modified cells. Such genetically modified cells may be syngeneic, allogeneic, or xenogeneic. Such genetically modified cells may produce a biologic for local delivery.

The methods of the present invention may be used for the localized delivery of an angiogenic factor. Such an angiogenic factor may be an anti-angiogenic factor, preventing or inhibiting local vascularization, including, for example, tumor vascularization. Alternatively, such an angiogenic factor may be pro-angiogenic factor, such as, for example, vascular endothelial growth factors C and D, promoting local angiogenesis of blood vessels and/or local lymphangiogenesis of lymphatic vessels (see, for example, Szuba et al., FASEB J. 2002 16(14):1985-7).

The methods of the present invention may be used for the localized delivery of an agent for the treatment of a bacterial, viral, and/or fungal infection. Methods for the localized delivery of an agent to a lymphatic capillary bed may be used for the treatment and/or prevention of a disorder of the lymphatic system such as lymphatic obstruction, bacterial infections, and parasitic infections, including, but not limited to, filariasis, loiasis, onchocerciasis, and dracunculiasis.

The methods of the present invention may be used for the diagnosis, staging, and/or treatment of cancer. For example, the methods of the present invention may be used for the localized delivery of a diagnostic agent, including, but not limited to a dye or an antibody to a tumor marker, including antibodies attached to a detectable marker. The delivery device may be located in close proximity to a tumor site and may deliver a marker or dye to assist in the identification of the sentinel lymph node draining a tumor site. As used herein, a “sentinel lymph node” is the first lymph node encountered by lymphatic vessels draining a tumor. The tumor status of the sentinel lymph node may be predictive of metastasis to the adjacent lymph nodes and the absence of tumor cells in the sentinel lymph node is believed to be indicative of the absence of metastatic disease in other lymph nodes and tissues. For example, the assessment of the sentinel lymph node(s) nearest to the site of the primary tumor lesion is an important staging and prognostic modality in the management of many different cancers, including, for example, malignant melanoma, breast cancer, colorectal cancer, prostate cancer, and non-small lung cell carcinoma. See, for example, Yudd et al., Radiographics, 1999, 19(2):343-56; Ajekigbe and Baguley, J R Coll Surg Edinb 2000, 45(6):382-5; Melfi et al., Eur J Cardiothorac Surg 2003, 23(2):214-20; Mariani et al., J Surg Oncol 2004, 85(3):141-51; Mariani et al., J Surg Oncol 2004, 85(3):112-22; and Braat et al., Dis Colon Rectum 2005, 48(2):371-83.

The methods of the present invention may be used for the localized delivery of a therapeutic agent for the treatment of cancer, including, for example, the delivery of a therapeutic agent to the lymphatic capillary bed draining a tumor site. Such delivery may be before, during, and/or after surgical excision of a tumor. Such localized delivery may allow for delivery at doses that are higher than doses tolerated when affected by systemic delivery.

The methods of the present invention may be used for the localized delivery of a vaccine. For example, a tumor vaccine may be delivered by the methods of the present invention. Tumor vaccines containing a specific protein of the tumor cell can be used to stimulate an immune response. Different types of vaccines are used to treat different types of cancer, including, for example, melanoma, myeloma, pancreatic cancer, prostate cancer, breast cancer, colon cancer, kidney cancer, lung cancer, or lymphoma. With the methods of the present invention, a tumor vaccine may be administered to the lymphatic capillary bed in which the tumor is located and/or to the lymphatic capillary bed that drains the tumor.

The methods of the present invention may be used for the localized delivery of a genetic material. Genetic materials are substances intended to have a direct or indirect genetic therapeutic effect and include, for example, genetic vectors, genetic regulator elements, genetic structural elements, DNA, small interfering RNA (also referred to as “siRNA,” see, for example Dorsett and Tuschl, siRNAs: Applications in Functional Genomics and Potential as Therapeutics,” Nat Rev Drug Discov, 2004, 3(4):318-29, and Ambion Technical Bulletin #506, “siRNA Design Guidelines) and the like.

The methods of the present invention may be used for the localized delivery of a visualization agent in methods of lymphoscintography, used, for example, in the evaluation of lymphedema and the identification of sentinel lymph nodes. See, for example, Yudd et al., Radiographics, 1999, 19(2):343-56; Ajekigbe and Baguley, J R Coll Surg Edinb 2000, 45(6):382-5; Melfi et al., Eur J Cardiothorac Surg, 2003, 23(2):214-20; Szuba et al., J Nucl Med, 2003, 44(1):43-57; Mariani et al., J Surg Oncol, 2004, 85(3):141-51; and Mariani et al., J Surg Oncol, 2004, 85(3):112-22.

The methods of the present invention may be used for the localized delivery of an agent to the lymphatic system at any of a variety of locations in a subject's body. A delivery device may, for example, be located subcutaneously, intraperitoneally, or intradermally. In some aspects of the methods of the present invention, the location of the delivery device and/or the delivery of the agent may be to the intradermal compartment of a subject's skin. See, for example, WO 2005/016401. In some aspects of the methods of the present invention, the location of the delivery device and/or the delivery of the agent is not to the intradermal compartment of a subject's skin. In some aspects of the methods of the present invention, the delivery of the agent may be by syringe or needle based technologies. See, for example, WO 2005/016401. In some aspects of the methods of the present invention, the delivery of the agent is not by syringe or needle based technologies.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Acute and Chronic Delivery of Dyes

The effect of local continuous delivery of dyes in a rat model system was used to map lymphatic drainage and to determine the feasibility of local delivery to lymphatic drainage beds. Animals were implanted with Alzet® pumps delivering tracking compounds for fourteen days. Dye diffusion was documented by photographing the viscera of animals at the termination of the study. The results demonstrate local delivery of tracking compounds without systemic distribution. Continuous local delivery of India ink was limited in distribution to the ipsilateral lymph node. India ink was not detected in the contralateral and distal lymphoid organs. Thus, the continuous targeted delivery of compounds to a target organ can be accomplished with minimal systemic side effects.

In this example the primary and secondary draining lymph nodes for two putative implant sites in rats were identified. The primary and secondary draining lymph nodes following acute administration of tracking dye were identified. And, the primary and secondary draining lymph nodes following chronic/continuous administration of tracking dye and/or protein were identified. Further, the drainage pattern from both a subcutaneous site (referred to herein as “SQ” or “SC”) and a site situated above the peritoneal membrane (IP/IP wall) were identified. For the purposes of this study, the designation “IP” refers to placement in the intraperitoneal wall and not to placement in the peritoneal cavity itself.

This example identified the draining lymph nodes in two different circumstances; when an acute bolus of dye was injected, and when a compound was continuously infused over a period of fourteen days.

Materials and Methods

Animals. Male Lewis Inbred rats, six to eight weeks of age, were obtained from Charles River Laboratories and were acclimatized for five days before use. Animals were anesthetized with sodium pentobarbital before implants and dye injections.

Dyes. Isosulfan blue, india ink, and goat anti rabbit horse radish peroxidase (HRP) were used.

Acute delivery. 40 microliters (μl) of isosulfan blue was injected subcutaneously and above the IP wall. The location of the injection was varied to detect the site which would drain into the para-aortic lymph nodes. Dye diffusion was monitored at five minutes and thirty minutes. The animals were then sacrificed and a gross visual examination of the internal organs was performed to identify dye drainage into internal organs.

Chronic delivery. Alzet® miniosmotic pumps with a delivery rate of 0.25 or 0.5 microliter/hour (μl/hr) were used to continuously infuse dye(s) for fourteen days. The animals were sacrificed and a gross visual examination of the internal organs was performed to identify dye drainage into internal organs. Lymph nodes were collected, photographed, and analyzed as described in Table 1, below.

TABLE 1

| # | Duration | Dye | Data collection |
|---|---|---|---|
| 1 | Acute, 5 and 30 | Isosulfan blue | Photographs |
| | mins | | |
| 2 | Chronic, 14 | Isosulfan blue + | Photographs + OPD* |
| | days | Goat anti-rabbit HRP | substrate development |
| | | | to reveal HRP** |
| 3 | Chronic, 14 | India Ink | Photographs + |
| | days | | histology and H&E*** |
| | | | staining |
| 3a | Chronic, 14 | India Ink | Photographs**** |
| | days | | |

*O-phenylenediamine (OPD)
**Horse radish peroxidase (HRP)
***hematoxylin and eosin staining (H&E)
****Confirmatory study to increase number of animals studied.

Results and Discussion

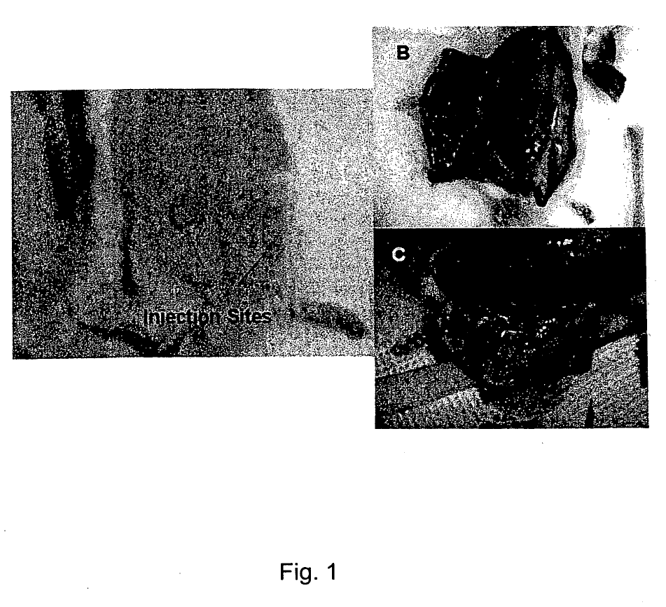

Acute Study/Subcutaneous injection. Minimal diffusion was observed with injection of dye at the site noted (see FIG. 1A, 1B, and 1C). FIG. 1B and FIG. 1C depict the extent of dye diffusion at 30 minutes following subcutaneous injection with 40 μl of isosulfan blue. Varying the site position (higher injection point as shown by the left injection mark in FIG. 1A) or the dorsal and ventral positions showed similar slow diffusion. An injection site 1 cm above the hip joint covered the inguinal and the popliteal beds as shown in FIG. 1B and FIG. 1C, with no internal organs being stained.

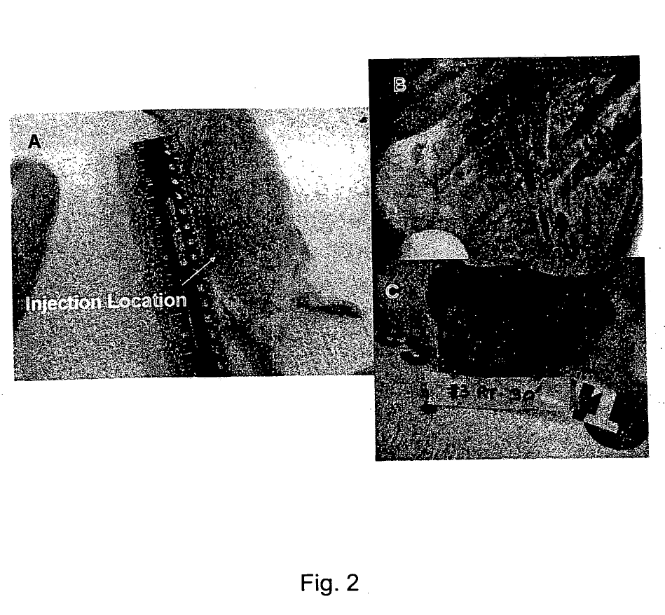

Acute Study/Peritoneal wall injection. An injection into the peritoneal wall one centimeter above the hip joint, as shown in FIG. 2A, covered the inguinal (FIG. 2B) and the popliteal beds (FIG. 2C), with no internal organs being stained. Similar to the data obtained from subcutaneous injections, minimal diffusion was observed with injection of the dye into the peritoneal wall (as shown in FIGS. 2B and 2C). The dye diffusion distance was however more that the subcutaneous injection. In one instance, the injection was directly into a blood vessel and in this case, internal organs were stained.

Acute dye delivery demonstrated that 1) dye diffusion was slow, 2) dye diffusion was localized to the immediate vicinity of the injection site, and 3) barring an actual injection into a blood vessel, the dye did not end up in circulation of the animal at a visually detectable concentration.

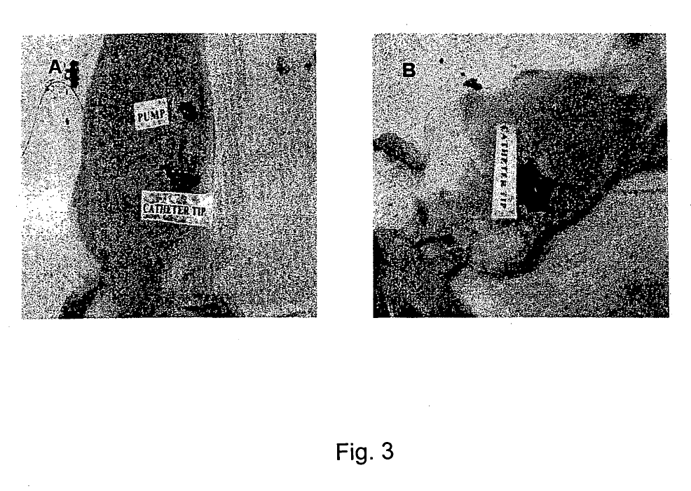

Based on the fact that systemic dosing was not occurring, Alzet® pumps with isosulfan blue and goat anti-rabbit HRP were implanted for continuous infusion for fourteen days. The implant sites were as depicted in FIG. 3A-3B.

With the first attempt of local delivery used an open ended delivery catheter connected to a twenty-eight day Alzet® pump pumping isosulfan blue and goat anti-rabbit HRP, the tip became occluded. Histology confirmed the ingrowth of tissue into the polyethylene catheter. Dye was not observed in any of the lymph nodes at 14 days.

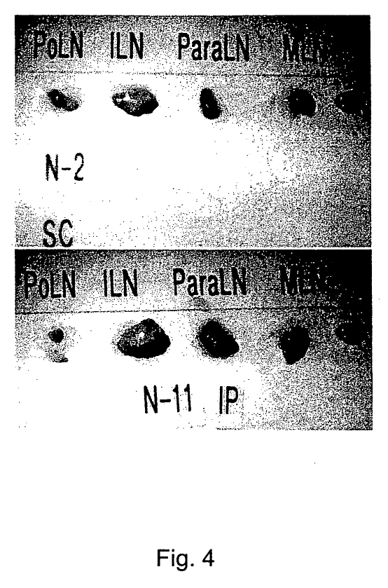

As shown in FIG. 4A-4C, the lymph nodes however showed peroxidase activity consistent with diffusion of molecules observed after acute delivery into the local lymphatic bed. The popliteal (labeled PoLN) and the para-aortic lymph nodes (labeled ParaLN) show higher peroxidase activity as compared to control lymph node with some activity in the mesenteric (labeled MLN) and the inguinal lymph nodes labeled (ILN). However, the data was not considered conclusive as red blood cells and other cell types have been demonstrated to have high peroxidase activity and the differences in staining could be attributed to a difference in the blood flow to the lymph nodes.

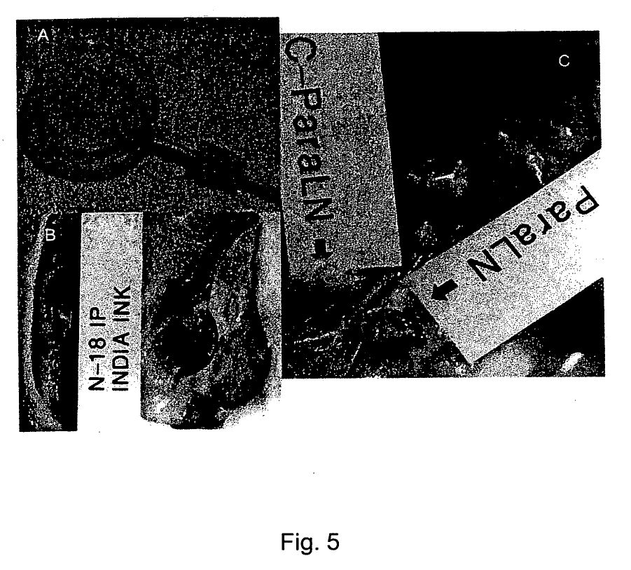

Therefore, India ink (colloidal carbon black), a non-biodegradable marker, was included. In addition to this change, a spacer capable of retarding the occlusion of the catheter was added (catheter shown in FIG. 5A). As shown in FIG. 5C, the primary draining site was identified as the ipsilateral para-aortic lymph node and no staining was observed in the contralateral para-aortic lymph node.

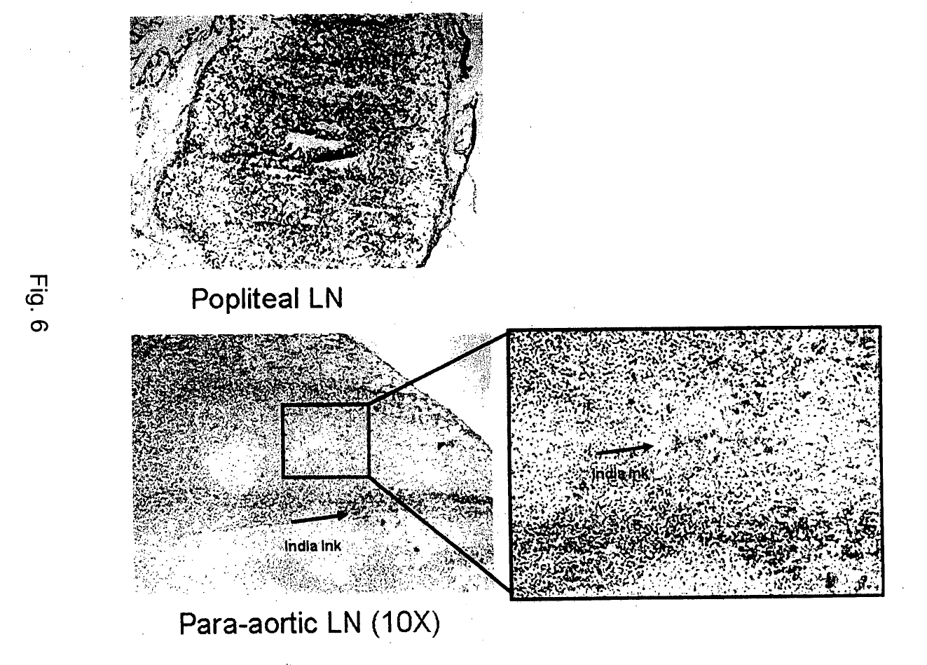

This was further confirmed by histology as shown in FIG. 6. The popliteal lymph node did not show any carbon deposition. The para-aortic lymph node showed discrete patches of carbon black. Some carbon deposition was found in the mesenteric lymph nodes and none in the inguinal lymph nodes.

This example presents the acute mapping of drainage beds identified the primary and secondary draining lymph nodes following subcutaneous or peritoneal wall delivery of a tracking dye. In this example chronic administration of isosulfan blue dye was unsuccessful, chronic administration of a tracking antibody indicated local delivery pattern similar to acute administration, and chronic administration of India ink conclusively showed that local drainage was specific to the point of discriminating between ipsilateral and contralateral lymph nodes.

This example demonstrates that it is possible to physically localize the delivery of compounds using acute and more importantly chronic delivery. A secondary demonstration was the delivery of HRP conjugated antibody, which trafficked to the lymph nodes even in the presence of fibrosis of the catheter tip. Taken together these results indicate that it is possible to chronically administer antibodies or small molecules to a local lymphatic organ without obvious systemic side effects. This study led to Example 2, the determination that the physical localization of an immunosuppressive therapy results in a functional difference in the response of lymphocytes.

### Example 2

### Localized Delivery of Immunosuppression: Antibody

In this example, the effect of local continuous delivery of an immunosuppressant was evaluated in a rat model system. Animals were implanted with Alzet® pumps delivering a lymphocyte depleting antibody, and monitored for systemic changes in lymphocyte numbers. Following twenty days of compound delivery, the animals were immunized with a foreign protein, and allowed time to respond to the protein. Lymphoid tissues were collected at the end of the study and analyzed for cell numbers, cell proliferation in response to immunized protein, and for their ability to secrete cytokines.

Materials and Methods

Animals and Dosing. Male Lewis inbred rats six to eight weeks of age were obtained from Charles River Laboratories and acclimated for five days before study initiation.

Commercially prepared complete Freund's adjuvant (CFA) was mixed with ovalbumin (200 milligram/milliliter (mg/ml) in phopshate buffered saline (PBS)) as a 1:1 volume/volume (v/v) emulsion with a two-glass syringe hub interlocked together. Using a glass syringe with a locking hub and a 26 gauge needle, animals were injected with 100 ml ovalbumin/CFA emulsion intradernally at the base of the tail. There were three groups in the pilot study: 1) a control group; 2) a group treated with anti-lymphocyte antibody (RALS) (for flow cytometric analysis); and 3) a group immunized with ovalbumin/CFA (for proliferation assays). Group assignments for the main study are shown in Table 2.

TABLE 2
Main Study Group Assignments

| Immunosuppressive | | Immunized with CFA + |
|---|---|---|
| Factor | Unimmunized | Ovalbumin |
| Site 1 (SC) high dose | Group 1 | Group 2 |
| Site 1 (SC) low dose | Group 3 | Group 4 |
| Site 2 (IP) high dose | Group 5 | Group 6 |
| Site 2 (IP) low dose | Group 7 | Group 8 |
| Control (untreated) | Group 9 | Group 10 |
| Control (single dose | Group 11 | |
| antibody) | | |

Immunization and Pump Implant Methods. For pump implants, animals were anesthetized by intraperitoneal injection of sodium pentobarbital at a dose of 60 milligram/kilogram (mg/kg) body weight. Catheter and pump were positioned as described in FIG. 7. The catheter tip was anchored with nonabsorbable sutures (4.0 Prolene).

Blood Collection. Blood was collected from retro-orbital sinus under CO₂/O₂ anesthesia into heparin-coated tubes. For the pilot study, blood was collected on days 1, 2, 3, 4, and 5 and for the main study blood was collected at weeks 1, 2, 3, and 4. A total of 0.8-1 ml of blood per animal was drawn per time point. Rat 1 in group 10 was inadvertently killed by overdose of anesthetic at week 3 of the study; therefore, blood was not collected from this animal in week 4.

Tissue Collection. For the pilot study, animals were euthanized by overdose of pentobarbital on day 10, and spleen, popliteal, inguinal, para-aortic, iliac, and mesenteric lymph nodes were collected. For the main study, animals were euthanized on day 28 and the same tissues were collected. However the para-aortic and iliac lymph nodes from the ipsilateral side were pooled and the para-aortic and iliac lymph nodes from the contralateral side were pooled. All samples were collected into tubes containing ice-cold collection medium (serum-free IMDM supplemented with L-glutamine, penicillin, and streptomycin) and were processed within two hours. Rat 1 in group 10 was inadvertently killed by overdose of anesthetic at week three of the study; therefore, tissues were not collected from this animal.

Flow Cytometric Analysis. Whole blood was centrifuged (600×g) for 20 minutes and plasma was collected and stored at −80° C. Blood pellets were resuspended in 20 ml lysis buffer (160 mM ammonium chloride, 17 mM Tris, pH 7.65) and incubated for 10 minutes at room temperature. Cold PBS (10 ml) was then added and cells were centrifuged (10 min at 250×g), supernatants were decanted, and cells were resuspended in FACS buffer (PBS containing 1% BSA and 0.05% sodium azide). After an additional wash, cells were resuspended in FACS buffer and counted by hemacytometer, using trypan blue to assess viability. Cells were added to a round-bottom 96 well plate, washed once with FACS buffer, and Fc block was added at 0.5 mg/well. Following 5 minutes incubation, staining antibodies and isotype controls were added at 0.5 mg/well, and plates were incubated at 2-8° C. for 45 minutes. For biotinylated antibody, APC-coupled streptavidin was added at 0.25 mg/well. Plates were then washed twice with 200 ml FACS buffer and once with 200 ml PBS. After the final wash, cells were resuspended in 100 ml PBS, transferred to FACS tubes, and 1% paraformaldehyde was added at 500 ml/tube. Tubes were stored in the dark at 2-8° C. for up to two days prior to analysis by FACScalibur flow cytometer. Forward-and side-scatter gates were set to exclude debris, cell clumps and contaminating erythrocytes. Isotype controls were used to optimize PMT and compensation settings.

Processing of Tissue Samples for Proliferation Assay. Spleens from each animal were transferred to sterile Dounce homogenizer tubes containing 2 ml of wash medium (serum-free IMDM) and were homogenized with several gentle strokes of the pestle. Spleen cell suspensions were transferred to a sterile centrifuge tube and centrifuged (250×g for 10 minute at 4° C.). After aspiration of the supernatant, 6 ml of RBC lysis buffer was added. Following a 5 minute incubation at room temperature, approximately 6 ml PBS was added and the cells were centrifuged. Cells were then be resuspended in 5 ml PBS and 1 ml of the suspension was transferred to a separate tube containing 4 ml proliferation medium (IMDM supplemented with 10% FBS, L-glutamine, penicillin, streptomycin, and 10 mM β-mercaptoethanol). An aliquot of cells was counted by hemacytometer and viability was determined by trypan blue. To generate single-cell suspensions, lymph nodes were pushed through a sterile 70 micron cell strainer situated in one of the wells of a sterile 6-well plate containing 2 ml wash medium. Cells and washings from the strainer were collected into a sterile centrifuge tube, and centrifuged as described above. Cells were then washed once with 3 ml wash medium, and resuspended in proliferation medium. An aliquot of cells was counted as described above.

Proliferation Assay. Cell concentrations were adjusted to 3×10⁶ cells/ml using proliferation medium (for the pilot experiment, splenocytes were also tested at a cell density of 5×10⁶ cells/ml). Cells were then plated at 100 ml/well in designated wells of the 96-well round-bottom plates. Ovalbumin was diluted in proliferation medium and added at 100 ml/well into designated wells of each plate. Final concentrations of ovalbumin for the pilot experiment were 1000, 100, 10, 1, and 0.1 mg/ml and for the main experiment final concentrations were 1000, 316, 100, 31.6, and 10 mg/ml. Plates were placed into a 37° C. humidified incubator with 5% CO₂. Following 34-38 hours of incubation, 50 ml of supernatant was carefully removed from each well without disturbing the cells underneath and transferred to a sterile 96-well plate in exactly the same format. These transfer plates were then frozen. An equal volume of fresh proliferation medium was added to replenish the wells. Following 70-74 hours of total incubation, 3H-thymidine was added at 20 ml/well (50 mCi/ml). After an additional 16-20 hours of incubation, cells were harvested onto filter mats using a Tomtec Mach 2-96 cell harvester and counts per minute (cpm) were measured using a Wallac Betaplate liquid scintillation counter.

Results and Discussion

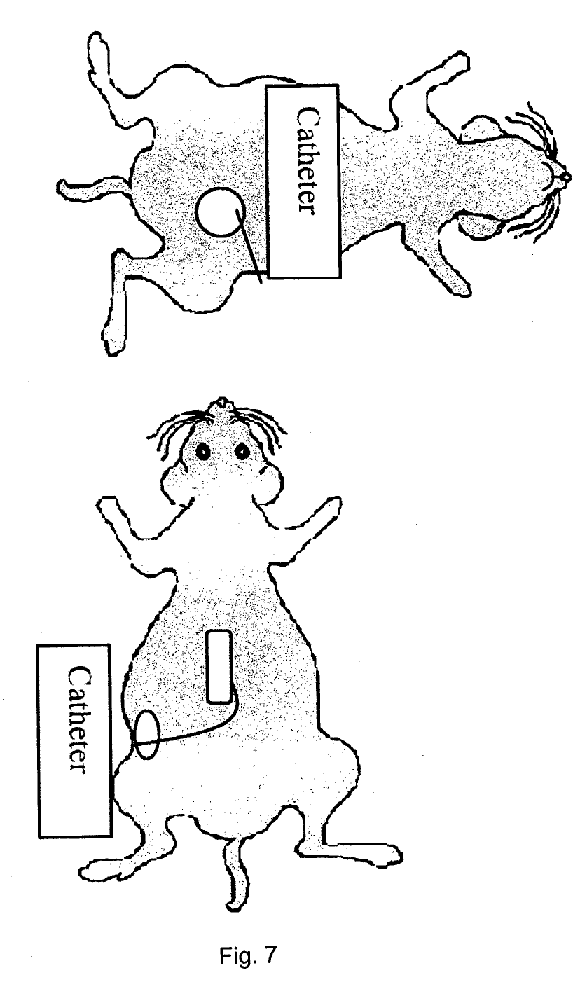

Flow Cytometry. There were no other large differences in relative cell numbers among the treatment groups from week 1 (FIG. 8A) and week 4 (FIG. 8B). As shown in FIG. 8, CD3⁺ T cells, Mac1⁺ macrophages and surface IgM⁺ Bcells were in similar relative ratios before and after treatment.

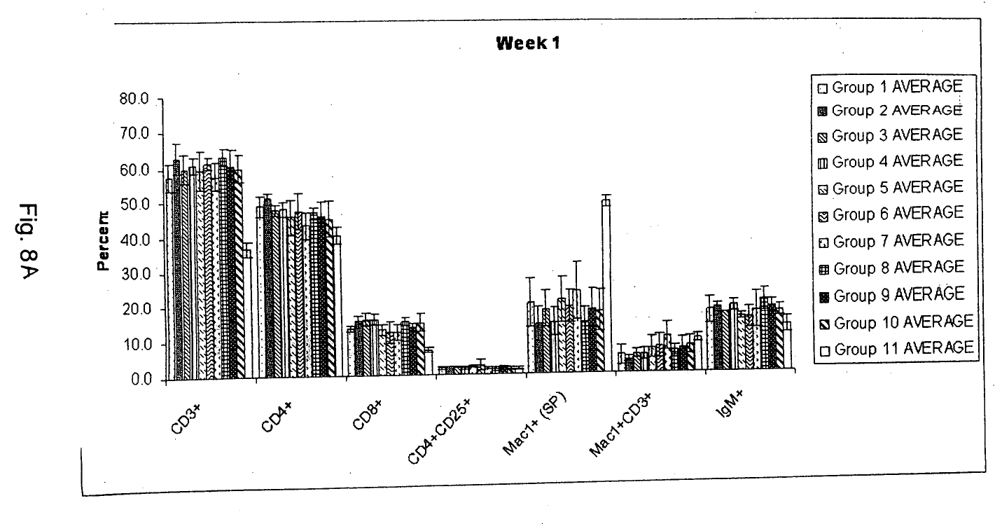

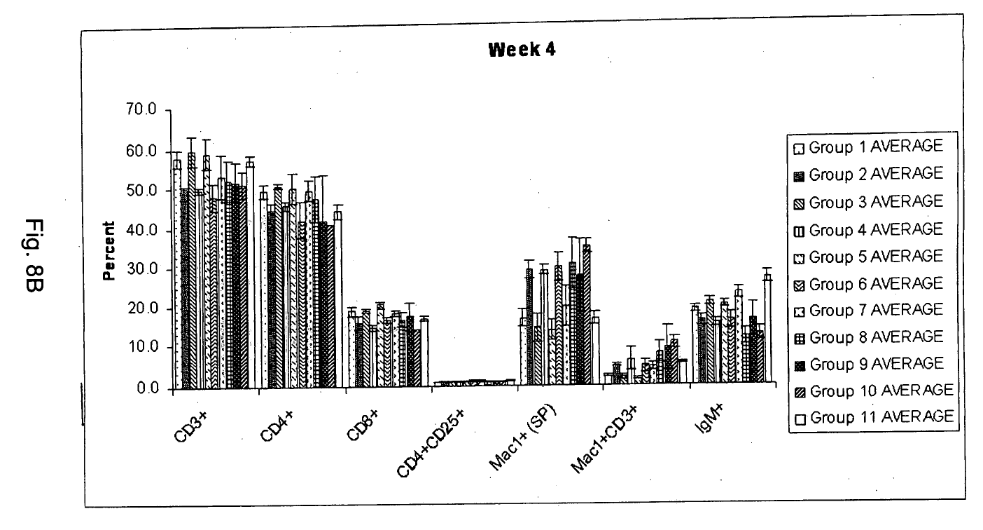

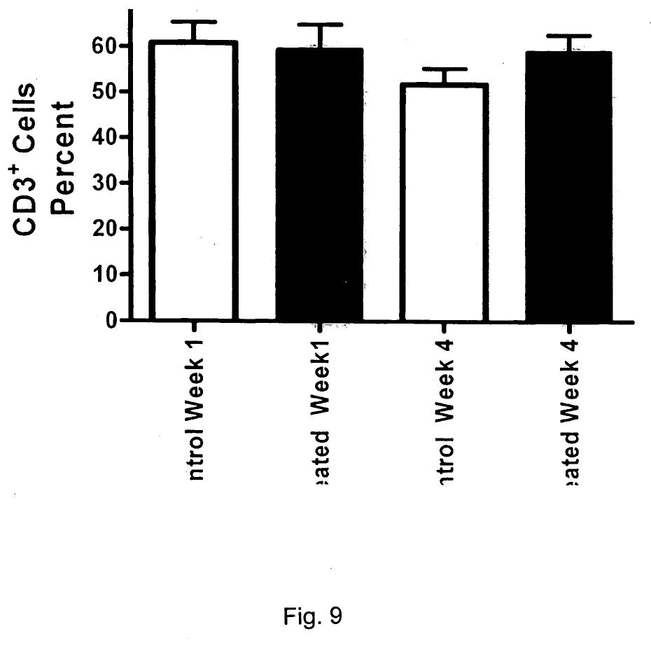

FIG. 9 depicts the ratios of CD3⁺ cells and confirms that cell ratios in peripheral blood were not altered by local treatment with rat anti-lymphocyte serum.

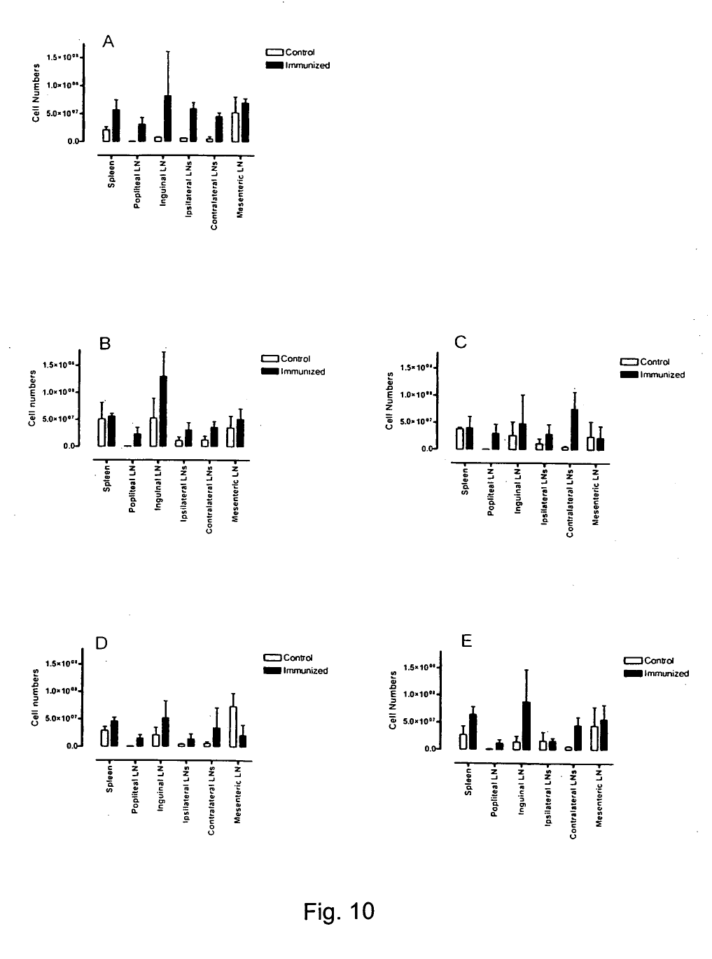

Proliferation. One objective of this study was to determine whether treatment with rat anti-lymphocyte serum (RALS) diminished the intensity of immune response generated when animals were immunized in the presence of local immunosuppression. Animals were immunized at the base of tail site, with an emulsion of ovalbumin in complete Freund's adjuvant while undergoing local immunosuppression. As shown in FIG. 10, cell numbers from the four treatment groups were compared. FIG. 10A represents data from animals that were not treated with anti-lymphocyte serum. The route of immunization (at the base of tail) increases the number of cells in the draining lymph nodes, the inguinal, ipsilateral and the contralateral paraaortic lymph nodes as shown by the dark bars. FIGS. 10B and 10C depict subcutaneous local delivery and FIGS. 10D and 10E depict the IP wall delivery of two doses of anti-lymphocyte serum. Compared to FIG. 10A, there is a reduction in cell number after immunization in all the groups, in the ipsilateral lymph nodes and a lesser decrease in the contralateral and inguinal lymph nodes. These data demonstrate that local delivery has a local effect.

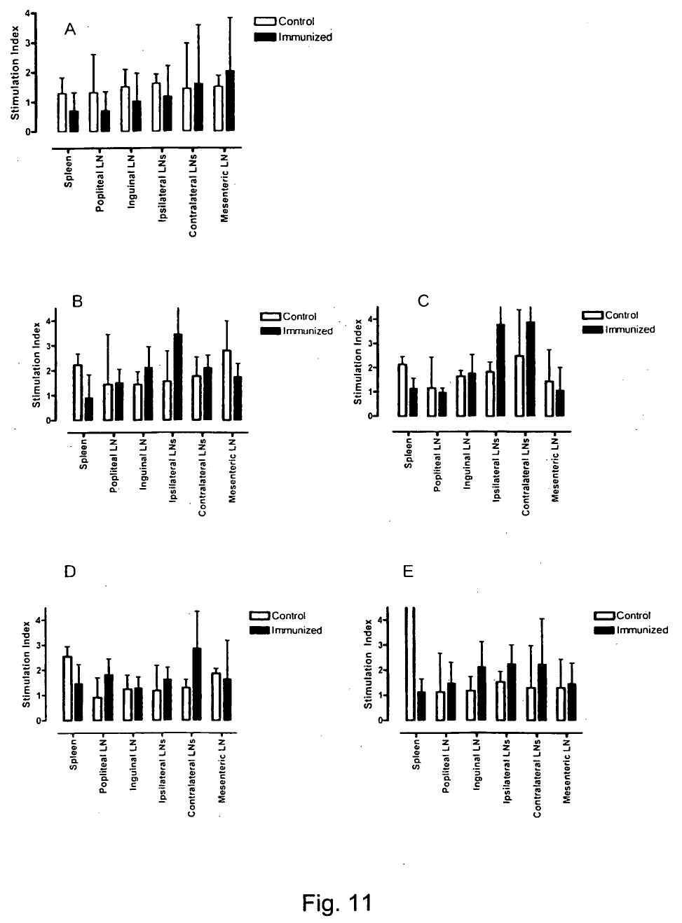

To determine whether, in addition to an effect on the cell numbers, the function of the cells is altered, lymphoid tissues from unimmunized and immunized animals were challenged with soluble ovalbumin for 72 hours to determine the proliferation of lymphocytes. Thirty-six hours after the challenge, supernatant was collected and analyzed for effector cytokines (Interferon-gama (IFN-γ)). FIG. 11A represents data from animals that were not treated with anti-lymphocyte serum. The route of immunization (at the base of tail) increases the number of cells in the draining lymph nodes and also their ability to proliferate when challenged with soluble ovalbumin, in vitro. FIG. 11B and 11C depict subcutaneous local delivery and FIG. 11D and 11E depict the IP wall delivery of two doses of anti-lymphocyte serum. The proliferation measured was not significantly different between the immunized and non-immunized groups, making further analysis difficult. However, the difference in the stimulation index of the IP-wall local immunosuppressed group in FIG. 11D showed a trend towards local immunosuppression as the immunized ipsilateral nodes showed a lesser degree of proliferation that the contralateral lymph nodes.

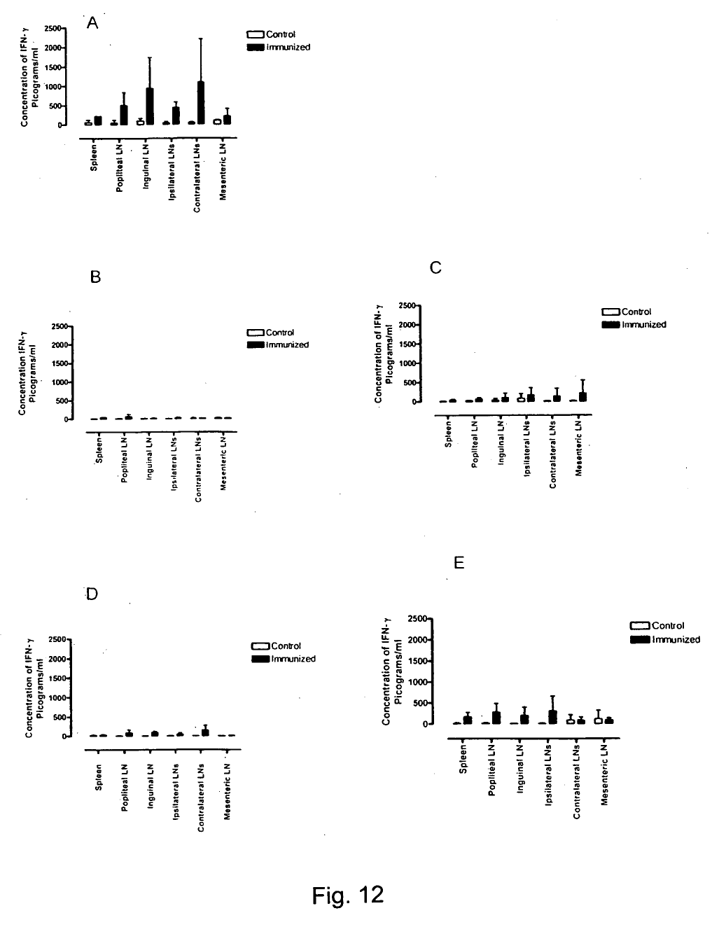

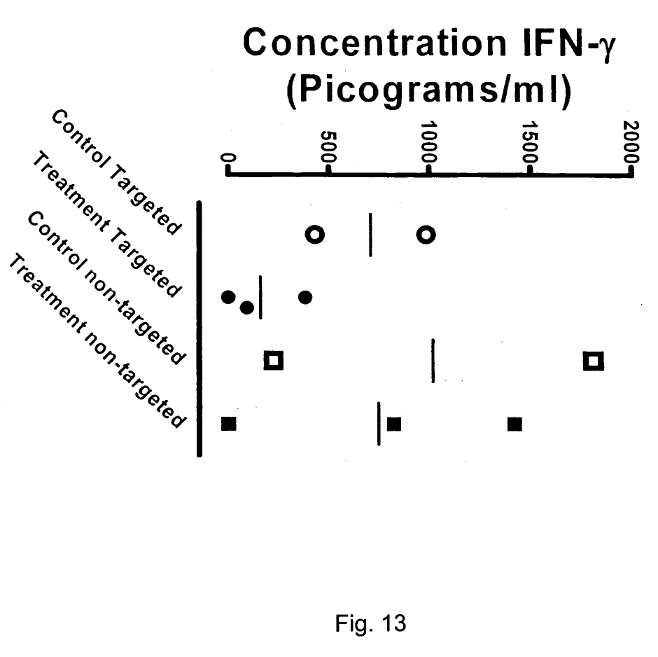

FIG. 12 depicts the secretion of effector cytokines from the same lymphocytes that showed marginal proliferation in FIG. 11. The data clearly show an increase in IFN-γ secretion in the untreated animals (FIG. 12A), and a lower level of IFN-γ in all the other groups tested. The dose of the immunosuppressive antibody seems to play a role in the intensity of the diminution of response. It is important to note that the spleen and the mesenteric lymph nodes show no increase in stimulation in FIG. 12A, and that the comparison be made only between the ipsilateral, contralateral and inguinal nodes. FIG. 13 shows the same data comparing within the IP-wall treated group (data rescaled from FIG. 12D) showing a difference between the targeted and untargeted lymph node response.

This example demonstrates that local immunosuppression is possible and that even when the global immunosuppressant anti-lymphocyte serum is used locally, a difference in the cell numbers and effector response can be observed.

### Example 3

### Localized Delivery of Immunosuppression: Small Molecules

In this example, the effect of local continuous delivery of immunosuppressive agents on immune responses was evaluated in a rat model system. Use of the immunosuppressive agents tested here served as a model for localized delivery of small molecules. Animals were implanted with Alzet® pumps delivering a “cocktail” of immunosuppressive agents, and immunized with a foreign protein. After induction of an immune response, cell proliferation was assayed in vitro by measuring thymidine [³H] incorporation. The lowest immunosuppressant dose having therapeutic efficacy was identified, and the distribution pattern of the effect on immune response following local delivery was established.

Materials and Methods

Animals. Male Lewis inbred rats six to eight weeks of age were obtained from Charles River Laboratories and acclimated for five days before study initiation. The study included a total of forty-two (42) animals, with 6 naïve animals (unimmunized control group) and 36 immunized animals (treatment groups).

Medical device implant. On day 1, all animals (n=42) received a medical device implant. Briefly, animals were anesthetized to by exposure to a mixture of isofluorane and oxygen by cone. Catheter and pump were positioned as shown in FIG. 7 as follows. With the animal in dorsal recumbency, a transverse cutaneous incision was made, a pocket was blunt-dissected, angled towards the left lower leg. A similar shorter pocket was extended towards the right shoulder to house the Alzet® pump, and the catheter was routed subcutaneously to the pump was on the back of the animal, as shown in FIG. 7. The skin was stapled/sutured with 6-0 viacryl.

Emulsion for immunization. Commercially prepared complete Freund's adjuvant (CFA) was mixed with ovalbumin as an emulsion in an interlocked two-glass-syringe hub. Ovalbumin was dissolved in PBS at 200 μg/ml, and then mixed with CFA to a final emulsion concentration of 100 μg OVA/PBS in 100 μl, with 100 μl CFA (i.e, 1:1 v/v), for a final volume of 200 μl. Rats were immunized by injection with ovalbumin/CFA emulsion intradermally at the base of the tail, using a glass syringe with a locking hub and a 26-G needle.

Dosing. On day 10 post-medical device implant, animals were dosed as shown in the study design presented in Table 3. In Group 1, unimmunized animals (n=6) were injected with PBS, and immunized animals (n-6) were injected with 100 μl of the ovalbumin/CFA emulsion. In Group 2, animals (n=6) were immunized by injection with 100 μl of the ovalbumin/CFA emulsion, and the implant site was treated with 6 μl/day of rabbit anti-reat lymphocyte serum at 15 mg/ml total (Cat. No. CL015A, CedarLane Laboratories, Burlington, N.C.). In Group 3, animals (n=6) were immunized by injection with 100 μl of the ovalbumin/CFA emulsion, and received a dose of a “cocktail” of rapamycin, leflunomide, and MPA at the highest concentration (see Table 4 for concentrations and calculated dose) by introduction of an Alzet® pumpt into the peritoneum without a catheter. In Group 4, animals (n=6) were immunized by injection with 100 μl of the ovalbumin/CFA emulsion, and received a “cocktail” of rapamycin, leflunomide, and MPA at the highest concentration (see Table 4) via the catheter tip placed subcutaneously under the inguinal fat bed. In Group 5, animals (n=6) were immunized by injection with 100 μl of the ovalbumin/CFA emulsion, and received a “cocktail” of rapamycin, leflunomide, and MPA at a medium concentration (see Table 4) via the catheter tip placed subcutaneously under the inguinal fat bed. In Group 6, animals (n=6) were immunized by injection with 100 μl of the ovalbumin/CFA emulsion, and received a “cocktail” of rapamycin, leflunomide, and MPA at the lowest concentration (see Table 4) via the catheter tip placed subcutaneously under the inguinal fat bed.

TABLE 3
Study design

| | Immunosuppressive | | Immunized | |
|---|---|---|---|---|
| | Factor Dosing via | Unimmu- | with CFA + | Number of |
| | Medical Device | nized | Chicken | Animals in |
| Group | Implant | Animals | ovalbumin | Each Group |
| 1 | Phosphate buffered | 6 | 6 | 12 |
| | saline (PBS) | | | |
| 2 | Anti-lymphocyte | | 6 | 6 |
| | antibody (ALS) | | | |
| 3 | “Cocktail” by | | 6 | 6 |
| | systemic delivery, | | | |
| | HIGH concentration | | | |
| 4 | “Cocktail” by | | 6 | 6 |
| | local delivery, | | | |
| | HIGH concentration | | | |
| 5 | “Cocktail” by | | 6 | 6 |
| | local delivery, | | | |
| | MEDIUM | | | |
| | concentration | | | |
| 6 | “Cocktail” by | | 6 | 6 |
| | local delivery, | | | |
| | LOW concentration. | | | |

| | | |
|---|---|---|
| Total: 6 | Total: 36 | Total: 42 |

The concentration of agents used in each treatment is set forth in Table 4 below. Based on the assumption that the average weight of each animal is 200 g, the daily dose for each treatment was calculated as shown in Table 4 below.

TABLE 4
Contents and calculated dose of each treatment

| | Immunosuppressive | | Dose/day |
|---|---|---|---|
| | Factor Dosing via | | (assume average |
| | Medical Device | | animal weight = |
| Group | Implant | Dose Contents | 200 g). |
| 1 | Phosphate buffered | | |
| | saline (PBS) | | |
| 2 | Anti-lymphocyte | Neat | |
| | antibody (ALS) | | |
| 3 | “Cocktail” by | Rapamycin 0.2 mg/kg | Rapamycin 40 μg |
| | systemic delivery, | Leflunomide 2 mg/kg | Leflunomide 400 μg |
| | HIGH concentration | MPA 4 mg/kg | MPA 800 μg |
| 4 | “Cocktail” by | Rapamycin 0.2 mg/kg | Rapamycin 40 μg |
| | local delivery, | Leflunomide 2 mg/kg | Leflunomide 400 μg |
| | HIGH concentration | MPA 4 mg/kg | MPA 800 μg |
| 5 | “Cocktail” by | Rapamycin 0.04 mg/kg | Rapamycin 8 μg |
| | local delivery, | Leflunomide 0.4 mg/kg | Leflunomide 80 μg |
| | MEDIUM | MPA 0.8 mg/kg | MPA 160 μg |
| | concentration | | |
| 6 | “Cocktail” by | Rapamycin 0.02 mg/kg | Rapamycin 4 μg |
| | local delivery, | Leflunomide 0.2 mg/kg | Leflunomide 40 μg |
| | LOW concentration. | MPA 0.4 mg/kg | MPA 80 μg |

Collection of blood samples. Whole blood was obtained on Days 0, 7, 10, 14, and at the termination of the study on Day 20 post-medical device implant. Data from Day 0 provided baseline measurements for the experiment. Blood was taken under 2-5% anesthesia from the retro-orbital plexus (˜0.5-1.0 ml of blood) of each animal. The whole blood samples were shipped in CytoChex storage and transport solution (Streck Laboratories, Omaha Nebr.) Dr. S. Pakala at Medtronic Inc. (Minneapolis Minn.) for flow cytometric analysis. Flow cytometry was performed on the whole blood samples to determine cell numbers and ratios of lymphocyte subsets. Blood serum was collected on Day 10 (i.e., prior to immunization with ovalbumin/CFA) and at the termination of the study on Day 20, using procedures, instructions, and tubes provided by Medtronic, Inc. The blood was stored at −4° C. and shipped to Dr. S. Pakala at Medtronic, Inc., for cytokine analysis.

Collection of tissue samples for assays. At the end of the study (Day 20 post-medical device implant), animals were euthanized with a carbon dioxide (CO₂ gas) overdose, and the tissue samples to be used for the cell proliferation assay were immediately collected from all animals and processed. Tissue was collected from the spleen, ispilateral lymph node, contralateral lymph node, mesenteric lymph node, and inguinal lymph node. Spleens and lymph nodes were collected from rats that were injected with ovalbumin/CFA on Day 10 to Day 14 (i.e., injected at some time between Day 10 and Day 14 post-medical device implant), into tubes containing 1 ml of serum-free Iscove's Modified Dulbecco's Medium (IMDM) supplemented with L-glutamine, penicillin and streptomycin (wash medium). Tissues were homogenized as described below, and T-cells from spleen and lymph nodes were used for in vitro proliferation assays using ³H-thymidine to measure proliferation.

Processing of spleen tissue. Spleen tissue was transferred to a sterile Dounce homogenizer tube (Kendall small tissue grinder, VWR Cat # 15704-126) containing 2 ml wash medium, and homogenized with 3-4 gentle strokes of the pestle. The spleen cell suspension was transferred to a 15-ml tube. The homogenizer was then washed with 3 ml wash medium and the wash was also added it to the tube. The cells were centrifuged at 400×g for 10 minutes at room temperature. The supernatant was aspirated and the pellet was then resuspended in 3 ml of RBC lysis buffer (0.16 M ammonium chloride, 0.01 M potassium bicarbonate, 0.096 mM EDTA and incubated at room temperature for 2 minutes. Ten (10) ml wash medium was added to dilute the lysis buffer, and the cells were centrifuged at 400×g for 10 minutes at room temperature. The supernatant was aspirated and the pellet was resuspended in 5 ml wash medium. Cells were triturated, allowing larger clumps of tissue to stick to the sides of the pipette. An additional 5 ml wash medium was added to the tube, and the cells were centrifuged at 400×g for 10 minutes at room temperature. Cells were resuspended in proliferation medium (Iscove's Modified Dulbecco's Medium supplemented with 10% FBS, L-glutamine, glutamine, Pen-strep and 10 μM β-mercaptoethanol). An aliquot of resuspended cells used to determine cell numbers and viability by counting in 0.25% trypan blue.

Proliferation assay for spleen cells. Cell concentration was adjusted to 3×10⁶ cells/ml in proliferation medium, and 0.1 ml of each spleen cell suspension (approximately 3×10⁵ cells/well) was plated in each designated well of a 96-well flat-bottom plate, leaving the first two rows of the plate empty. Plates were incubated at 37° C., 7.5% CO₂ in a humidified incubator until addition of ovalbumin. Ovalbumin was diluted from 2 mg/ml to a concentration of 0.01 mg/ml (1000 μg/ml) and further diluted 5 times to produce solutions having concentrations of 316 μg/ml ovalbumin, 100 μg/ml ovalbumin, 31.6 μg/ml ovalbumin, 10 μg/ml ovalbumin, and 3.16 μg/ml ovalbumin. Each solution was added to four (4) wells, providing 4 replicates of each treatment. The plates were incubated at 37° C., 7.5% CO₂ for 72 hours in a humidified incubator. During incubation, at 36 hours, 50 μl of supernatant was removed from each well without disturbing the cells underneath, and transferred to a sterile 96-well plate in exactly the same format. These supernatants were frozen and shipped to Medtronic, Inc. for further testing.

A working solution of ³H thymidine was prepared by diluting stock ³H thymidine having a specific activity of approximately 6.7 Ci/mmol, in a sufficient amount of proliferation medium to give a working solution of 50 μCi/ml ³H thymidine. After 72-hours of incubation, each well containing cells was pulsed with 1 μCi ³H-thymidine, and the plate was incubated for an additional 18-24 hours. Cells were harvested onto filter mats using the TOMTEC Harvester (Tomtec, Hamden Conn.), and radioactvity on filter mats (dpm) was measured using a Wallac Betaplate Liquid Scintillation Counter. Counts were recorded and the data was analyzed using appropriate statistical analyses.

Processing of lymph nodes. Lymph nodes were collected in a tube containing 1 ml wash medium and then transferred to a sterile cell strainer (70 micron nylon) in a 60 mm sterile petri dish containing an additional 3 ml wash medium was added. Cells were dispersed from the lymph nodes using a sterile 3 ml syringe plunger to gently push on the nodes, allowing cells to pass through the strainer into the petri dish. Cells were collected into a sterile 15-ml conical-end tube, after which the strainer and plunger were washed with 3 ml wash medium and the wash was added to the 15-ml tube. Cells were centrifuged at 400×g for 8 minutes at room temperature and the supernatant was aspirated. Cells were washed with 5 ml wash medium to remove fragments, centrifuged at 400×g for 8 minutes at room temperature, and the supernatant was aspirated. The cell pellet was resuspended in 2 ml proliferation medium for cell counting. For bigger lymph nodes, the resuspended cells were diluted 1:10 in trypan blue and then counted. For smaller lymph nodes, the resuspended cells were diluted 1:5 in trypan blue and then counted. Cell counts were performed on lymph node cell preparations from control (unimmunized) animals first, followed by counts of lymph node cell preparations from immune-activated animals.

Proliferation assay for lymph node cells. Cell concentration was adjusted to 3×10⁶ cells/ml in proliferation medium, and 0.1 ml of each lymph node cell suspension (approximately 3×10⁵ cells/well) was plated in each designated well of a 96-well flat-bottom plate, leaving the first two rows of the plate empty. Plates were incubated at 37° C., 7.5% CO₂ in a humidified incubator until addition of ovalbumin. Ovalbumin was diluted from 2 mg/ml to a concentration of 0.01 mg/ml (1000 μg/ml) and further diluted 5 times to produce solutions having concentrations of 316 μg/ml ovalbumin, 100 μg/ml ovalbumin, 31.6 μg/ml ovalbumin, 10 μg/ml ovalbumin, and 3.16 μg/ml ovalbumin. Each solution was added to four (4) wells, providing 4 replicates of each treatment. The plates were incubated at 37° C., 7.5% CO₂ for 72 hours in a humidified incubator. During incubation, at 36 hours, 50 μl of supernatant was removed from each well without disturbing the cells underneath, and transferred to a sterile 96-well plate in exactly the same format. These supernatants were frozen and shipped to Medtronic, Inc. for further testing.

A working solution of ³H thymidine was prepared by diluting stock ³H thymidine having a specific activity of approximately 6.7 Ci/mmol, in a sufficient amount of proliferation medium to give a working solution of 50 μCi/ml ³H thymidine. After 72-hours of incubation, each well containing cells was pulsed with 1 μCi ³H-thymidine, and the plate was incubated for an additional 18-24 hours. Cells were harvested onto filter mats using the TOMTEC Harvester (Tomtec, Hamden Conn.), and radioactvity on filter mats (dpm) was measured using a Wallac Betaplate Liquid Scintillation Counter. Counts were recorded and the data was analyzed using appropriate statistical analyses.

Cytokine assays. Assays for the effector cytokines interferon gamma (IFN-γ) and tumor necrosis factor alpha (TNFα) are performed on the serum samples and the supernatants collected at 36 hours as described above. Levels of the cytokine interleukin-2 (IL-2) in samples collected at 36 hours are measured using an ELISA assay, and the results used to corroborate the data from the proliferation assays.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

All headings are for the convenience of the reader and should not be used to limit the meaning of the text that follows the heading, unless so specified.

What is claimed is:

1. A method for the localized delivery of an agent to the lymphatic system of a subject, the method comprising providing the subject with a delivery device and delivering the agent via the delivery device, wherein the agent is delivered into a lymphatic capillary bed.

2. The method of claim 1, wherein the delivery device has a variable delivery rate.

3. The method of claim 1, wherein delivery of the agent by the device is active.

4. The method of claim 1, wherein the delivery device is implanted in the subject.

5. A method for the localized delivery of an agent to the lymphatic system of a subject, the method comprising providing a delivery device to the subject and delivering the agent via the delivery device, wherein the delivery of the agent by the delivery device is active, variable, implantable, or combinations thereof, and wherein the agent is delivered into a lymphatic capillary bed.

6. A method for limiting the distribution of an agent in a subject's body, the method comprising providing the subject with a delivery device and delivering the agent via the delivery device, wherein the agent is delivered into a lymphatic capillary bed.

7. The method of claim 1, wherein the agent is delivered to one or more lymph nodes of the lymphatic capillary bed.

8. The method of claim 1, wherein the delivery device is located subcutaneously.

9. The method of claim 1, wherein the delivery device is located intraperitoneally.

10. The method of claim 1, wherein the agent is delivered to the surface of a solid organ.

11. The method of claim 1, wherein the agent is delivered to the peritoneal membrane.

12. The method of claim 1, wherein the agent is an immunomodulator.

13. The method of claim 1, wherein the agent is an immunosuppressant.

14. The method of claim 1, wherein the agent is a hormone or growth factor.

15. The method of claim 1, wherein the agent is an angiogenic factor.

16. The method of claim 1, wherein delivery is to a site from which a tumor has been surgically excised.

17. The method of claim 1, wherein the subject has been the recipient of a cell, tissue, or organ transplant.

18. The method of claim 17, wherein the cell, tissue, or organ transplant is located within the lymphatic capillary bed.

19. The method of claim 17, wherein the cell, tissue, or organ transplant is selected from the group consisting of pancreatic islet cells, cardiac cells, muscle cells, a heart, a heart valve, liver, lung, kidney, and pancreas.

20. The method of claim 17, wherein the cell, tissue, or organ transplant has been genetically modified to express a therapeutic agent.

21. The method of claim 1, wherein the delivery device delivers a cell, tissue or organ transplant.

22. The method of claim 1, wherein the delivery of the agent by the delivery device is active, variable, implantable, or combinations thereof.

23. A method for limiting the distribution of an agent in a subject's body comprising localized delivery of an agent to the lymphatic system of a subject according to the method of claim 1.

24. A method of preventing or treating grant versus host disease comprising localized delivery of an immunosuppressant according to the method of claim 17.

25. The method of claim 6, wherein the delivery of the agent by the delivery device is active, variable, implantable, or combinations thereof.
